(19) United States
(12) Patent Application Publication
(10) Pub. No.: US 2017/0322225 A1
(43) Pub. Date: Nov. 9, 2017

(54) BIOMARKERS AND METHODS OF PREDICTION

(71) Applicant: Hoffmann-La Roche Inc., Little Falls, NJ (US)

(72) Inventors: Thomas Dieterle, Freiburg (DE); Eric J. Niesor, Nyon (CH); Rosemarie Kientsch-Engel, Feldafing (DE); Horst Klima, Penzberg (CH); Fabian Model, Rheinfelden (DE); Gabriela Bucklar, Binningen (CH); Vinzent Rolny, Muenchen (DE)

(73) Assignee: Hoffmann-La Roche Inc., Little Falls, NJ (US)

(21) Appl. No.: 15/493,661

(22) Filed: Apr. 21, 2017

Related U.S. Application Data
(63) Continuation of application No. PCT/EP2015/074242, filed on Oct. 20, 2015.

(30) Foreign Application Priority Data
Oct. 22, 2014 (EP) 14189840.3

(51) Int. Cl. G01N 33/68; G01N 33/74

(52) U.S. Cl. CPC G01N 33/6893; G01N 33/74; G01N 2800/324; G01N 2333/4737; G01N 2800/50; G01N 2333/58

(57) ABSTRACT

Subject of the present invention are biomarkers and methods for the identification of risk for subsequent cardiovascular event (e.g. coronary heart disease death, non-fatal myocardial infarction, ischemic stroke, hospitalizations for unstable angina pectotis, cardiac arrest) in patients that have experienced an acute coronary syndrome, comprising the detecting the level of NT-proBNP, homocysteine and CRP.

### CROSS-REFERENCE TO RELATED APPLICATIONS

This application is a continuation of International Patent Application No. PCT/EP2015/074242, having an international filing date of Oct. 20, 2015, the entire contents of which are incorporated herein by reference, and which claims benefit under 35 U.S.C. §119 to European Patent Application No. 14189840.3, filed on Oct. 22, 2014.

### SEQUENCE LISTING

The instant application contains a Sequence Listing which has been submitted electronically in ASCII format and is hereby incorporated by reference in its entirety. Said ASCII copy, created on Apr. 10, 2017, is named P32368-US SequenceListing.txt and is 3,470 bytes in size.

### FIELD OF THE INVENTION

The field of the invention relates to identify a population susceptible to have an increased risk of experiencing a cardiovascular event, such as cardiovascular death, non-fatal myocardial infarction, ischemic stroke, repeated hospitalizations for unstable angina pectoris, coronary revascularization or cardiac arrest, following a coronary heart disease (CHD), particularly following an acute coronary syndrome (ACS), more particularly following a recent ACS.

### BACKGROUND OF THE INVENTION

Despite achieving targets of low-density lipoprotein (LDL) cholesterol, blood pressure, and blood glucose through aggressive pharmacological treatment of these cardiovascular risk factors, patients that have experienced an acute coronary syndrome remain at high risk for suffering from a secondary cardiovascular event such as death from coronary heart disease, non-fatal myocardial infarction, ischemic stroke, repeated hospitalizations for unstable angina pectoris or cardiac arrest. The remaining risk despite optimal therapy of cardiovascular risk factors is termed “residual cardiovascular risk”. In a meta-analysis comprising nearly 29,000 patients with cardiovascular diseases (CVD) in 14 randomized trials comparing statin to no statin, 21.2 percent of treated patients experienced a major cardiovascular event during five years of follow-up (Cholesterol Treatment Trialists' (CTT) Collaborators. Efficacy and safety of cholesterol-lowering treatment: prospective meta-analysis of data from 90056 patients in 14 randomized trials of statins. The Lancet 2005; 366: 1267-1278.). With 1.14 million of patients being discharged from hospitals with a primary or secondary diagnosis of ACS every year in the USA alone, there is a high unmet medical need to improve post-ACS care including both diagnosis and treatment of residual risk (Heart Disease and Stroke Statistics—2014 Update: A report from the American Heart Association. Circulation 2014; 129: e28-e292.).

Identification of patients at high residual risk is mandatory to individually tailor the timing and the amount of clinical patient monitoring visits as well as future diagnostic and therapeutic interventions. Published risk scores such as HEART, TIMI, or GRACE were developed for application in the acute situation in patients with ACS at the time of admission to the hospital. They provide information about 6-months mortality risk (GRACE) respectively the risk for secondary cardiovascular events within 24 h to 6 weeks (HEART, TIMI) (GRACE: Fox, K A A, et al. BMJ 2006; 333: 1091-1094.; HEART: Backus, B E, et al. Int J Cardio 2013; 168: 2153-2158.; TIMI: Wiviott, S D, et al. J Am Coll Cardiol 2006; 47: 1553-1558.). However, these scores were not designed to predict individual residual risk in stable patients under optimal therapy for the prevention of secondary cardiovascular events that have recently experienced an ACS and therefore are useless for application in this patient population. Due to the lack of clinically applicable tools for risk stratification, national and international guidelines recommend aggressive treatment of cardiovascular risk factors in patients after ACS with acetylsalicylic acid (ASA), statins, beta-blockers, and angiotensin converting enzyme (ACE) inhibitors/angiotensin receptor blockers where applicable. No guidelines exist regarding the frequency and timing of clinical monitoring. Moreover, the frequency and timing of stress testing procedures respectively invasive diagnostic procedures such as coronary angiography are not clear though early detection of progression of the underlying disease would allow for earlier treatment with subsequent beneficial effects on outcome.

Therefore, there is a high unmet medical need for an easy-to-use clinical tool that allows the identification of patients at high risk for secondary cardiovascular events after an acute coronary syndrome.

### BRIEF DESCRIPTION OF THE FIGURES

FIG. 1: Prognostic Model Comparison (MBS is the 3 markers model: homcysteine, proBNP, CRP)

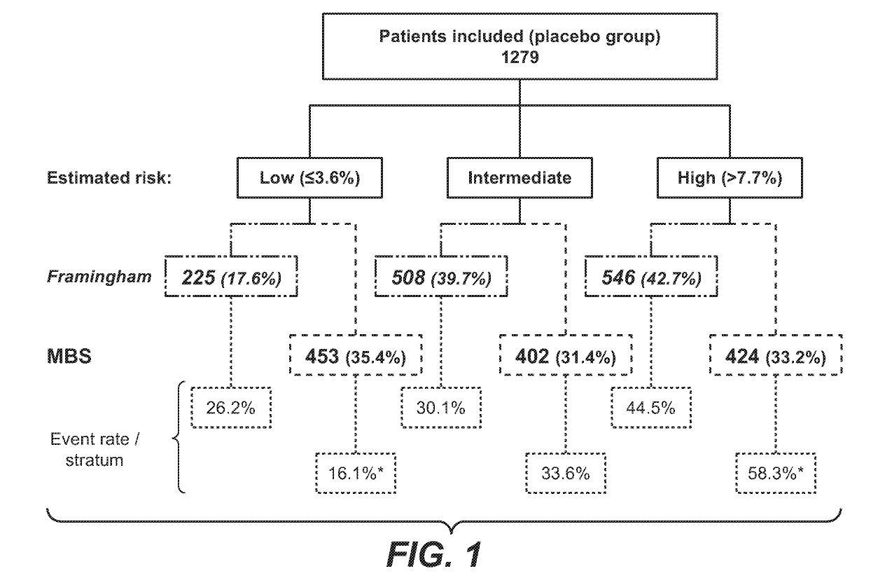

### DETAILED DESCRIPTION OF THE INVENTION

The present disclosure provides methods for identification of a subject suffering from stable Coronary heart disease (CHD), particularly with a documented recent Acute Coronary Syndrome (ACS), more particularly a post Acute Coronary Syndrome (ACS), most particularly a recent ACS, as having an increased risk of a cardiovascular event, in particular an other cardiovascular event, more particularly a secondary cardiovascular event.

The present disclosure provides methods for identification of a subject as having an increased risk of experiencing a cardiovascular event, following a acute coronary syndrome, more particularly following a recent acute coronary syndrome.

The present disclosure provides methods for identification of a subject as having an increased risk of experiencing a another cardiovascular event, more particularly a secondary cardiovascular event, following an acute coronary syndrome, more particularly following a recent acute coronary syndrome.

One aspect of the invention provides a method for identifying a subject suffering from stable Coronary heart disease (CHD), particularly with a documented recent Acute Coronary Syndrome (ACS), more particularly a post Acute Coronary Syndrome (ACS), most particularly a recent ACS, as having an increased risk of a cardiovascular event, particularly an other cardiovascular event, more particularly a secondary cardiovascular event, the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

A second aspect of the invention provides a method for identifying a subject suffering from stable Coronary heart disease (CHD), particularly with a documented recent Acute Coronary Syndrome (ACS), more particularly a post Acute Coronary Syndrome (ACS), most particularly a recent ACS, as having an increased risk of a cardiovascular event, particularly an other cardiovascular event, more particularly a secondary cardiovascular event, the method consisting of:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

An other aspect of the invention provides a method for identifying a subject suffering from a recent ACS, as having an increased risk of a secondary cardiovascular event the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a secondary cardiovascular event, if the amount of NT-proBNP, homocysteine and CRP in the sample is greater than the reference amount of NT-proBNP, homocysteine and CRP.

An other aspect of the invention provides a method for identifying a subject suffering from a recent ACS, as having an increased risk of a secondary cardiovascular event the method consisting of:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a secondary cardiovascular event, if the amount of NT-proBNP, homocysteine and CRP in the sample is greater than the reference amount of NT-proBNP, homocysteine and CRP.

An other aspect of the invention provides a method for identifying a subject as having an increased risk of experiencing a cardiovascular event, particularly an other cardiovascular event, in particular a secondary cardiovascular event, following an acute coronary syndrome, more particularly following a recent acute coronary syndrome, the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

An other aspect of the invention provides a method for identifying a subject as having an increased risk of experiencing a cardiovascular event, particularly an other cardiovascular event, in particular a secondary cardiovascular event, following an acute coronary syndrome, more particularly following a recent acute coronary syndrome, the method consisting of:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

An other aspect of the invention provides a method for identifying a subject as having an increased risk of experiencing a secondary cardiovascular event, following a recent acute coronary syndrome, the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a secondary cardiovascular event, if the amount of NT-proBNP, homocysteine and CRP in the sample is greater than the reference amount of NT-proBNP, homocysteine and CRP.

An other aspect of the invention provides a method for identifying a subject as having an increased risk of experiencing a secondary cardiovascular event, following a recent acute coronary syndrome, the method consisting of:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a secondary cardiovascular event, if the amount of NT-proBNP, homocysteine and CRP in the sample is greater han the reference amount of NT-proBNP, homocysteine and CRP.

In certain embodiments, the invention provides the method as described herein, wherein the cardiovascular event is selected from cardiovascular death, non-fatal myocardial infarction (MI), non-fatal stroke of ischemic origin, hospitalization for unstable angina, coronary revascularization and cardiac arrest.

In certain embodiments of the above aspect, the detecting comprises contacting, in vitro, the sample with a combination of detection agents, each agent having specific binding affinity for one of the biomarkers.

In certain embodiments of the above aspect, the agent is an antibody or fragment thereof.

In certain embodiments of the above aspect, the sample is blood, plasma, serum or urine, more particularly from blood, plasma or serum, most particularly blood.

In certain embodiments of the above aspect, the subject is identified as having an increased risk, particularly low (less than 3.6%) or high risk (more than 7.7%), more particularly high risk (more than 7.7%) of a cardiovascular event, particularly an other cardiovascular event, more particularly a secondary cardiovascular event, when the amounts of the NT-proBNP, homocysteine and CRP in the sample are greater than the median of their respective reference amount.

In certain embodiments of the above aspect, the subject is identified as having an increased risk of a cardiovascular event, particularly an other cardiovascular event, more particularly a secondary cardiovascular event, when the amount of the NT-proBNP, homocysteine and CRP in the sample is in the fourth quartile range of their respective reference amount.

In certain embodiments of the above aspect, the method further comprises the step of recommending a therapy to treat cardiovascular disease, if the subject is identified as having an increased risk of a cardiovascular event, particularly an other cardiovascular event, in particular a secondary cardiovascular event.

In more particular embodiments of the above aspect, the method further comprises the step of recommending a therapy to treat cardiovascular disease, if the subject is identified as having an increased risk of a secondary cardiovascular event.

In certain embodiments of the above aspect, the method further comprises the step of administering to the subject a pharmaceutical agent to treat cardiovascular disease, if the subject is identified as having an increased risk of a cardiovascular event, particularly an other cardiovascular event, in particular a secondary cardiovascular event.

In more particular embodiments of the above aspect, the method further comprises the step of administering to the subject a pharmaceutical agent to treat cardiovascular disease, if the subject is identified as having an increased risk of a secondary cardiovascular event.

In certain embodiments of the above aspect, the therapy comprises an investigational new drug therapy.

In another embodiment, the application discloses a device adapted for carrying out the method as above described comprising:

a) an analysing unit comprising a combination of detection agents which specifically bind to NT-proBNP, homocysteine and CRP, the analysing unit adapted for contacting, in vitro, the sample from the subject with the detection agent;
b) an evaluation unit including a computing device having a database and a computer-implemented algorithm on the database, the computer-implemented algorithm when executed by the computing device determines an amount of the biomarker in the sample from the subject and compares the determined amount of NT-proBNP, homocysteine and CRP with the corresponding NT-proBNP, homocysteine and CRP reference amount and provides a diagnosis of at increased risk of a cardiovascular event, particularly an other cardiovascular event, more particularly a secondary cardiovascular event if the amount of the NT-proBNP, homocysteine and CRP determined in the step of determining is greater than the corresponding NT-proBNP, homocysteine and CRP references amount.

In another embodiment, the application discloses a device adapted for carrying out the method as above described consisting of:

a) an analysing unit comprising a combination of detection agents which specifically bind to NT-proBNP, homocysteine and CRP, the analysing unit adapted for contacting, in vitro, the sample from the subject with the detection agent;

b) an evaluation unit including a computing device having a database and a computer-implemented algorithm on the database, the computer-implemented algorithm when executed by the computing device determines an amount of the biomarker in the sample from the subject and compares the determined amount of NT-proBNP, homocysteine and CRP with the corresponding NT-proBNP, homocysteine and CRP reference amount and provides a diagnosis of at increased risk of a cardiovascular event, particularly an other cardiovascular event, more particularly a secondary cardiovascular event if the amount of the NT-proBNP, homocysteine and CRP determined in the step of determining is greater than the corresponding NT-proBNP, homocysteine and CRP references amount.

In a particular embodiment of the device as defined herein, wherein the database further includes the NT-proBNP, homocysteine and CRP references amount.

In another embodiment, the application discloses a kit adapted for carrying out the method as described herein, comprising detection agents for NT-proBNP, homocysteine and CRP and instructions for carrying out the method.

In a particular embodiment, the kit as herein described further comprises a combination of detection agents for NT-proBNP, homocysteine and CRP.

In certain embodiments of the above aspects, the detecting comprises contacting, in vitro, the sample with a combination of detection agents, each agent having specific binding affinity for one of the biomarkers. In certain embodiments, the agent is antibody or fragment thereof.

In certain embodiments of the above aspects, the sample is a serum or blood sample.

In certain embodiments of the above aspects, the subject is identified as having an increased risk of disease progression when the amount of the biomarkers in the sample is greater than the median of the reference amount. In certain embodiments of the above aspects, the subject is identified as having an increased risk of disease progression when the amount of the biomarkers in the sample is in the fourth quartile range of the reference amount.

Another aspect of the invention provides for a device adapted for carrying out the method of any of the proceeding claims comprising: a) an analysing unit comprising a combination of detection agents which specifically bind to the biomarkers, the analysing unit adapted for contacting, in vitro, the sample from the subject with the detection agent; b) an evaluation unit including a computing device having a database and a computer-implemented algorithm on the database, the computer-implemented algorithm when executed by the computing device determines an amount of the biomarker in the sample from the subject and compares the determined amount of the biomarker with a biomarker reference amount and provides a diagnosis of at increased risk for disease progression if the amount of the biomarker determined in the step of determining is greater than the biomarker reference amount. In one embodiment, the database further includes the biomarker reference amount.

Another aspect of the invention provides for a device adapted for carrying out the method of any of the proceeding claims consisting of: a) an analysing unit comprising a combination of detection agents which specifically bind to the biomarkers, the analysing unit adapted for contacting, in vitro, the sample from the subject with the detection agent; b) an evaluation unit including a computing device having a database and a computer-implemented algorithm on the database, the computer-implemented algorithm when executed by the computing device determines an amount of the biomarker in the sample from the subject and compares the determined amount of the biomarker with a biomarker reference amount and provides a diagnosis of at increased risk for disease progression if the amount of the biomarker determined in the step of determining is greater than the biomarker reference amount. In one embodiment, the database further includes the biomarker reference amount.

Another aspect of the invention provides for a kit adapted for carrying out the method of any of the proceeding claims, comprising a detection agent for the biomarkers and instructions for carrying out the method. In one embodiment, the kit further comprises a combination of detection agents for the biomarkers.

The terms “N-terminal of the pro-hormone brain natriuretic peptide” or “NT-proBNP” refer to Amino-terminal proBNP, exemplified by SEQ ID NO: 1, (Swiss Prot Accession Number NP_002512.1, Gene ID NCBI 4879), WO 02/089657, WO 02/083913, EP 0 648 228. “NT-proBNP” encompasses the protein having the amino acid sequence of SEQ ID NO: 1 as well as variants, homologues and isoforms thereof. Such variants, homologues and isoforms have at least the same essential biological and immunological properties as the specific NT-proBNP. For example, they share the same essential biological and immunological properties if they are detectable by the same specific assays referred to in this specification, e.g., by ELISA assays using polyclonal or monoclonal antibodies specifically recognizing the NT-proBNP polypeptides. Exemplary assays are described in the accompanying Examples. Variants referred to above may be allelic variants or any other species specific homologs, paralogs, or orthologs. Moreover, the variants referred to herein include fragments of the specific NT-proBNP polypeptides or the aforementioned types of variants as long as these fragments have the essential immunological and biological properties as referred to above. Such fragments may be, e.g., degradation products of the NT-proBNP polypeptides. Further included are variants which differ due to posttranslational modifications such as phosphorylation or myristylation

The term “homocysteine” is produced within cells by the metabolism of methionine from dietary protein. Intracellular concentrations are kept by export into the plasma, where it becomes oxidized rapidly and circulates as one of three forms (Table 1). The parameter measured most frequently in clinical laboratories is the combined sum of all three forms, which is referred to as “total homocysteine”. According to the present invention “total homocysteine” and “homocysteine” should be understood as interchangeable. Indeed homocysteine level being measure according to the present invention is the total homocysteine level according to Table 1.

TABLE 1
the three forms of homocysteine present within the circulation

| | Protein-homocysteine miced | Homocysteine-cysteine mixed | |
|---|---|---|---|
| | disulphide | disulphide | homocysteine |
| Structure | 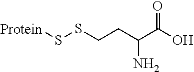 | 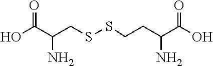 [SMILES: NC(CCSSCC(N)C(=O)O)C(=O)O] 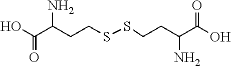 [SMILES: NC(CCSSCCC(N)C(=O)O)C(=O)O] | 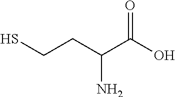 |
| Proportion | 70-90% | 8-19% | <2% |
| of Total | | | |

| | | |
|---|---|---|
| Terminology | Protein-bound | Non protein-bound (free) |

Total homocysteine

Reference: package insert Roche Homocysteine enzymatic assay for Cobas®, Roche Diagnostics International Ltd

The terms “C-reactive protein” or “CRP” refers to an annular pentameric protein found on the first chromosome. exemplified by SEQ ID NO: 2 (Swiss Prot Accession number NP_000558). According to the invention, a high-sensitivity CRP (hs-CRP) is recommend to be used to determine the concentration of CRP. The hs-CRP test measures low levels of CRP using laser nephelometry. The advantage of using such method is the speed and the high sensitivity

The term “increased risk of experiencing a cardiovascular event ” as used herein means that the subject to be analyzed by the method of the present disclosure is allocated either into the group of subjects of a population having a low (i.e., non-elevated) risk for experiencing a cardiovascular event or into a group of subjects having a significantly elevated risk, high risk group. An increased risk as referred to in accordance with the present disclosure means that the risk of experiencing a cardiovascular event within a predetermined predictive window is elevated significantly for a subject with respect to the average risk for disease progression in a population of subjects.

The term “aptamer” refers to oligonucleotides, including RNA, DNA and RNA/DNA molecules, or peptide molecules, which exhibit the desired biological activity, in particular, binding to the corresponding target molecule.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, bronchial lavage or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation. In particular, the sample refers to a sample of body fluid from samples of blood, plasma, serum or urine, more particularly from blood, plasma or serum.

The term “diagnosing” or “identifying” or “assessing” as used herein means predicting whether the risk for a “residual cardiovascular risk” or of experiencing another cardiovascular event, is increased in a subject after a cardiovascular event, more particularly in a recent cardiovascular event, or not. As will be understood by those skilled in the art, such a prediction is usually not intended to be correct for 100% of the subjects to be diagnosed. The term, however, requires that the prediction to be at increased risk for disease progression, or not, is correct for a statistically significant portion of the subjects (e.g. a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Example confidence intervals are at least 90%, at least 95%, at least 97%, at least 98% or at least 99%. The p-values include 0.1, 0.05, 0.01, 0.005, or 0.0001.

The phrase “provides a diagnosis/assessment” as used herein refers to using the information or data generated relating to the level or presence of the biomarker(s) in a sample of a patient to diagnose/assess the risk of “residual cardiovascular risk” or of experiencing another cardiovascular event in the patient. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of the biomarker(s) to a reference level. In some embodiments, the information or data includes an indication that the biomarker(s) is present or absent in the sample. In some embodiments, the information or data includes an indication that the patient is diagnosed/assessed with an increased risk of “residual cardiovascular risk” or of experiencing another cardiovascular event.

The term “detecting” the amount of a biomarker peptide or polypeptide as used herein refers to measuring the amount or concentration, semi-quantitatively or quantitatively for example. Measuring can be done directly or indirectly, more particularly directly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

The term “subject” as used herein relates to animals, such as mammals (for example, humans). The subject according to the present disclosure shall suffer from cardiovascular disease, stable cardiovascular disease or acute coronary syndrome as described elsewhere herein.

“Cardiovascular events” as used herein refers to cardiovascular death, non-fatal myocardial infarction (MI), non-fatal stroke of ischemic origin, hospitalization for unstable angina and coronary revascularization.

The term “comparing” as used herein refers to comparing the level of the biomarker in the sample from the individual or patient with the reference level of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device (e.g., of a system disclosed herein). The value of the measured or detected level of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references which are stored in a database by a computer program. The computer program may further evaluate the result of the comparison, i.e. automatically provide the desired assessment in a suitable output format. For a computer assisted comparison, the value of the determined amount may be compared to values corresponding to suitable references which are stored in a database by a computer program. The computer program may further evaluate the result of the comparison, i.e. automatically provides the desired assessment in a suitable output format.

The term “reference amount” as used herein refers to an amount which allows assessing whether a subject suffering from cardiovascular disease has an increased risk of a cardiovascular event. The reference may e.g. be derived from a pool of subjects from the general population who have not suffered from any cardiovascular event. Moreover, the reference amount may define a threshold amount or range, whereby dependent on the type of reference a change in the determined amount with respect to the threshold is either indicative for an increased risk for disease progression or a normal risk. Alternatively, an essentially identical amount may be either indicative for an increased risk for disease progression or a normal risk as well, if a suitable reference amount is used. The reference amount applicable for an individual subject may vary depending on various physiological parameters such as age, gender, or subpopulation, as well as on the means used for the determination of the polypeptide or peptide referred to herein. A suitable reference amount may be determined from a reference sample to be analyzed together, i.e. simultaneously or subsequently, with the test sample.

The term “binding agent” refers to a molecule that comprises a binding moiety which specifically binds the corresponding target biomarker molecule. Examples of “binding agent” are a nucleic acid probe, nucleic acid primer, DNA molecule, RNA molecule, aptamer, antibody, antibody fragment, peptide, peptide nucleic acid (PNA) or chemical compound.

The term “probe” or “nucleic acid probe” refers to a nucleic acid molecule that is capable of hybridizing with a target nucleic acid molecule (e.g., genomic target nucleic acid molecule) and, when hybridized to the target, is capable of being detected either directly or indirectly. Thus probes permit the detection, and in some examples quantification, of a target nucleic acid molecule. In particular examples, a probe includes a plurality of nucleic acid molecules, which include binding regions derived from the target nucleic acid molecule and are thus capable of specifically hybridizing to at least a portion of the target nucleic acid molecule. A probe can be referred to as a “labeled nucleic acid probe,” indicating that the probe is coupled directly or indirectly to a detectable moiety or “label,” which renders the probe detectable.

The term “primer” or “nucleic acid primer” refers to a short single stranded polynucleotide, generally with a free 3′—OH group, which binds to a target molecule potentially present in a sample of interest by hybridizing with a target sequence, and thereafter promotes polymerization of a polynucleotide complementary to the target.

The term “specific binding” or “specifically bind” refers to a binding reaction wherein binding pair molecules exhibit a binding to each other under conditions where they do not significantly bind to other molecules.

The term “specific binding” or “specifically binds”, when referring to a protein or peptide as a binding agent, refers to a binding reaction wherein a binding agent binds to the corresponding target molecule with an affinity of at least 10-7 M. The term “specific binding” or “specifically binds” preferably refers to an affinity of at least 10-8 M or even more preferred of at least 10-9 M for its target molecule. The term “specific” or “specifically” is used to indicate that other molecules present in the sample do not significantly bind to the binding agent specific for the target molecule. Preferably, the level of binding to a molecule other than the target molecule results in a binding affinity which is only 10% or less, more preferably only 5% or less of the affinity to the target molecule.

The term “specific binding” or “specifically binds”, when referring to a nucleic acid as a binding agent, refers to a hybridization reaction wherein a binding agent or a probe contains a hybridizing region exactly or substantially complementary to the target sequence of interest. A hybridization assay carried out using the binding agent or probe under sufficiently stringent hybridization conditions enables the selective detection of the specific target sequence. The hybridizing region is preferably from about 10 to about 35 nucleotides in length, more preferably from about 15 to about 35 nucleotides in length. The use of modified bases or base analogues which affect the hybridization stability, which are well known in the art, may enable the use of shorter or longer probes with comparable stability. A binding agent or a probe can either consist entirely of the hybridizing region or can contain additional features which allow for the detection or immobilization of the probe, but which do not significantly alter the hybridization characteristics of the hybridizing region.

The term “specific binding” or “specifically binds”, when referring to a nucleic acid aptamer as a binding agent, refers to a binding reaction wherein a nucleic acid aptamer binds to the corresponding target molecule with an affinity in the low nM to pM range.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “recent” as used herein refers to an event that occur in the past six months, more particularly up to three months. For instance “recent” according to the present invention, a recent event is an event that occur in the past three months.

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “device” as used herein relates to a system comprising the aforementioned units operatively linked to each other as to allow the diagnosis according to the methods of the disclosure. Example detection agents which can be used for the analyzing unit are disclosed elsewhere herein. The analyzing unit may comprise said detection agents in immobilized form on a solid support which is to be contacted to the sample comprising the biomarkers the amount of which is to be determined. Moreover, the analyzing unit can also comprise a detector which determines the amount of detection agent which is specifically bound to the biomarker(s). The determined amount can be transmitted to the evaluation unit. Said evaluation unit comprises a data processing element, such as a computer, with an implemented algorithm for carrying out a comparison between the determined amount and a suitable reference.

The term “kit” as used herein refers to a collection of the aforementioned components which may be provided separately or within a single container. The container also comprises instructions for carrying out the method of the present disclosure. These instructions may be in the form of a manual or may be provided by a computer program code which is capable of carrying out the comparisons referred to in the methods of the present disclosure and to establish a diagnosis accordingly when implemented on a computer or a data processing device. The computer program code may be provided on a data storage medium or device such as an optical storage medium (e.g., a Compact Disc) or directly on a computer or data processing device.

Clinical risk prediction models incorporate multiple variables to prognosticate the risk of adverse events for an individual patient. Age, pulse rate (PBM), LDL cholesterol level, arterial hypertension, diabetes, peripheral vascular disease, Congestive Heart Failure, previous acute coronary syndrome, previous revascularization, previous stroke, coronary heart disease, treatment with diuretics are strong risk factors for acute coronary syndrome. The biomarker approach described herein reflects the various pathways involved in the pathogenesis of cardiovascular events and provides a prediction of secondary cardiovascular events. The use and implementation of this approach can be used to identify segments of the CHD, in particular the acute coronary syndrome population that would benefit most from a novel treatment. Clinical use of the biomarkers and methods described herein is useful in identifying which patients may need a different treatment and avoid additional therapeutic options in patients with a lowest risk of progression. Furthermore, the present invention may benefit for a better diagnostic of the population at risk. Embodiments of the instant disclosure also encompass diagnostic devices and kits for carrying out the aforementioned methods.

One aspect of the present disclosure relates to methods for diagnosing whether a subject suffering from acute coronary syndrome or recent cardiovascular event is at increased risk of experiencing a cardiovascular event. In one embodiment, the method comprises detecting the amount of NT-proBNP, homocysteine and CRP biomarkers in a sample of the subject and comparing the amount to a reference. In particular, the method consist of detecting the amount of NT-proBNP, homocysteine and CRP biomarkers in a sample of the subject and comparing the amount to a reference. The subject is identified as having an increased risk of experiencing a cardiovascular event if the amount of the biomarkers in the sample is greater than the reference amount of the respective biomarkers. In a more particular embodiment the reference amounts are for CRP 1.51 mg/L, homocysteine 12.16 μmol/L, NT-proBNP 263 pg/ml.

Another aspect of the present disclosure relates to methods for monitoring whether a subject suffering from a post acute coronary syndrome is at increased risk of a cardiovascular event during the course of treatment for cardiovascular disease, in particular for acute coronary syndrome. In one embodiment, the method comprises detecting the amount of NT-proBNP, homocysteine and CRP biomarkers in a sample of the subject and comparing the amount to a reference. In particular, the method consists of detecting the amount of NT-proBNP, homocysteine and CRP biomarkers in a sample of the subject and comparing the amount to a reference. The subject is identified as having an increased risk of a cardiovascular event if the amount of the biomarkers in the sample is greater than the reference amount of the biomarkers. In one embodiment, the reference is from a sample of subjects not suffering from cardiovascular disease, in particular acute coronary syndrome. In another embodiment, the reference is sample taken from the subject prior to beginning a new additional treatment for cardiovascular disease, in particular for acute coronary syndrome or a sample taken from the subject at a timepoint during the new additional treatment process. The treatment may be modified based on the results of this method. For example, the treatment may be continued if the subject exhibits a decrease in the amount of biomarker(s) as compared to the reference. Conversely, the treatment may be substituted for an alternative treatment if the subject exhibits an increase in the amount of biomarker(s) as compared to the reference.

Another aspect of the invention relates to a device adapted for carrying out the methods provided above and herein is provided. Exemplary embodiments of the device comprise a) an analysing unit comprising a detection agent which specifically binds to a biomarker of the invention, said analysing unit adapted for contacting, in vitro, a portion of a sample from the subject with the detection agent; b) an evaluation unit including a computing device having a database and a computer-implemented algorithm on the database, the computer-implemented algorithm when executed by the computing device determines an amount of the biomarker in the sample from the subject and compares the determined amount of the biomarker with a biomarker reference amount and provides a diagnosis of at increased risk for disease progression if the amount of the biomarker determined in said step of determining is greater than the biomarker reference amount. According to some embodiments, the database further includes the biomarker reference amount.

Another aspect of the invention provides for a kit adapted for carrying out the above disclosed methods of the present disclosure comprising a detection agent for the biomarker(s) as well as instructions for carrying out the method. In one embodiment, the kit is for diagnosing whether a subject suffering from acute coronary syndrome, in particular a recent cardiovascular event, is at increased risk for experiencing an other cardiovascular event, in particular a secondary cardiovascular event.

In one embodiment, the amounts of the three biomarkers determined in the test sample are increased as compared to the reference amounts for the biomarkers is indicative for a subject who has an increased risk of experiencing an other cardiovascular event, in particular a secondary cardiovascular event.

In one embodiment, the amounts of all biomarkers markers, including the clinical biomarkers, determined in the test sample are increased as compared to the reference amounts for the biomarkers is indicative for a subject who has an increased risk of experiencing an other cardiovascular event, in particular a secondary cardiovascular event.

In one embodiment, the subject is identified as having an increased risk of experiencing an other cardiovascular event, in particular a secondary cardiovascular event, if the amount of the biomarkers determined in the test sample is greater than the reference amount. In one embodiment, the reference amount is the median amount derived from a cohort of subjects not suffering from cardiovascular disease, in particular form an acute coronary syndrome.

### Methods of Detecting the Biomarkers

Biomarkers, including proteins or nucleic acids, can be detected using methods generally known in the art. Methods of detection generally encompass methods to quantify the level of a biomarker in the sample (quantitative method) or that determine whether or not a biomarker is present in the sample (qualitative method). It is generally known to the skilled artisan which of the following methods are suitable for qualitative and/or for quantitative detection of a biomarker. Samples can be conveniently assayed for, e.g., proteins using Westerns and immunoassays, like enzyme-linked immunosorbent assays (ELISAs), Radioimmunoassays (RIAs), fluorescence-based immunoassays, as well as mRNAs or DNAs from a genetic biomarker of interest using Northern, dot-blot, polymerase chain reaction (PCR) analysis, array hybridization, RNase protection assay, or using DNA SNP chip microarrays, which are commercially available, including DNA microarray snapshots. Further suitable methods to detect biomarker include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys® analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used immunoassays.

Methods for measuring electrochemiluminescent phenomena are well-known. Such methods make use of the ability of special metal complexes to achieve, by means of oxidation, an excited state from which they decay to ground state, emitting electrochemiluminescence. For review see Richter, M. M., Chem. Rev. 104 (2004) 3003-3036.

Biomarkers can also be detected by generally known methods including magnetic resonance spectroscopy (NMR spectroscopy), Gas chromatography-mass spectrometry (GC-MS), Liquid chromatography-mass spectrometry (LC-MS), High and ultra-HPLC HPLC such as reverse phase HPLC, for example, ion-pairing HPLC with dual UV-wavelength detection, capillary electrophoresis with laser-induced fluorescence detection, anion exchange chromatography and fluorescent detection, thin layer chromatography.

In accordance with the present disclosure, detecting the amount of a biomarker peptide or polypeptide can be achieved by all known means for determining the amount of a peptide in a sample. Examples of such means include immunoassay devices and methods which may utilize labeled molecules in various sandwich, competition, or other assay formats. These assays will develop a signal which is indicative for the presence or absence of the peptide or polypeptide. Moreover, the signal strength can be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. These methods may comprise biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on Elecsys® analyzers), CBA (an enzymatic Cobalt Binding Assay, available for example on Roche-Hitachi.™. analyzers), and latex agglutination assays (available for example on Roche-Hitachi.™. analyzers).

According to the instant disclosure, determining the amount of a biomarker peptide or polypeptide may comprise the steps of (a) contacting a cell capable of eliciting a cellular response the intensity of which is indicative of the amount of the peptide or polypeptide with the said peptide or polypeptide for an adequate period of time, (b) measuring the cellular response. For measuring cellular responses, the sample or processed sample may be added to a cell culture and an internal or external cellular response is measured. The cellular response may include the measurable expression of a reporter gene or the secretion of a substance, e.g. a peptide, polypeptide, or a small molecule. The expression or substance shall generate an intensity signal which correlates to the amount of the peptide or polypeptide.

Also, detecting the amount of a biomarker peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Detecting the amount of a biomarker peptide or polypeptide may comprise the steps of (a) contacting the peptide with a specific ligand, (b) (optionally) removing non-bound ligand, (c) measuring the amount of bound ligand. The bound ligand will generate an intensity signal. Binding according to the present disclosure includes both covalent and non-covalent binding. A ligand according to the present disclosure can be any compound, e.g., a peptide, polypeptide, nucleic acid, or small molecule, binding to the peptide or polypeptide described herein. Exemplary ligands include antibodies, nucleic acids, peptides or polypeptides such as receptors or binding partners for the peptide or polypeptide and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab and F(ab).sub.2 fragments that are capable of binding antigen or hapten.

The present disclosure also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. The ligand or agent binds specifically to the peptide or polypeptide. Specific binding according to the present disclosure means that the ligand or agent should not bind substantially to (“cross-react” with) another peptide, polypeptide or substance present in the sample to be analyzed. The specifically bound peptide or polypeptide should be bound with at least 3 times higher, and in some embodiments at least 10 times higher or even at least 50 times higher affinity than any other relevant peptide or polypeptide. Non-specific binding may be tolerable, if it can still be distinguished and measured unequivocally, e.g. according to its size on a Western Blot, or by its relatively higher abundance in the sample. Binding of the ligand can be measured by any method known in the art. Said method may be semi-quantitative or quantitative. Suitable methods are described in the following.

First, binding of a ligand may be measured directly, e.g. by NMR or surface plasmon resonance. Second, if the ligand also serves as a substrate of an enzymatic activity of the peptide or polypeptide of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, the amount of substrate may be saturating. The substrate may also be labeled with a detectable label prior to the reaction. For example, the sample is contacted with the substrate for an adequate period of time. An adequate period of time refers to the time necessary for a detectable, and in some embodiments measurable, amount of product to be produced. Instead of measuring the amount of product, the time necessary for appearance of a given (e.g. detectable) amount of product can be measured. Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.).

The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag may be at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star.™. (Amersham Biosciences), ECF.™. (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include .sup.35 S, .sup.125 I, .sup.32 P, .sup.33 P and the like. A radioactive label can be detected by any method known and appropriate, e.g. a light-sensitive film or a phosphor imager. Suitable measurement methods according the present disclosure also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other detection methods as described above.

According to embodiments of the instant disclosure, the amount of a peptide or polypeptide may be detected as follows: (a) contacting a solid support comprising a ligand for the peptide or polypeptide as specified above with a sample comprising the peptide or polypeptide and (b) measuring the amount peptide or polypeptide which is bound to the support. The ligand may be chosen from the group consisting of nucleic acids, peptides, polypeptides, antibodies and aptamers. In some embodiments, the ligand is present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the disclosure. Suitable methods for fixing/immobilizing said ligand are well known and include, but are not limited to ionic, hydrophobic, covalent interactions and the like. It is also contemplated to use “suspension arrays” as arrays according to the present disclosure (Nolan 2002, Trends Biotechnol. 20(1): 9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

### Reference Amount:

Reference amounts can be calculated for a cohort of subjects (i.e. subjects which are known to have CHD) based on the average or mean values for a given biomarker by applying standard statistically methods. In one embodiment, the reference is determined in a cohort of subjects suffering from CHD using multivariable Proportional Hazard (Cox) Regression analysis (Cox DR. Regression models and life tables. J R Stat Soc (B). 1972; 34(series B): 187-220). Techniques and assays useful in this type of analysis are described in the example and figures referenced therein.

Table 2 provides the means and the median value calculated based on the data obtained according to example 1.

TABLE 2
Biomarkers hsCRP, homocysteine, NT-proBNP:
means, medians, inter-quartile ranges (IQR), units;

| | Placebo | | | |
|---|---|---|---|---|
| Biomarker | Mean +/− SD | Median [IQR] | N | Unit |
| hsCRP | 3.64 +/− 8.68 | 1.51 [0.81-3.61] | 7501 | mg/l |
| Homocysteine | 13.33 +/− 5.61 | 12.16 [9.99-14.98] | 2062 | μmol/l |
| NT-proBNP | 573 +/− 1329 | 263 [122-595] | 2072 | pg/ml |

The median values for the biomarker(s) determined in a cohort of patients may be also used as a basis for establishing reference levels.

In certain embodiments, the term “reference level” herein refers to a predetermined value. In this context “level” encompasses the absolute amount, the relative amount or concentration as well as any value or parameter which correlates thereto or can be derived therefrom. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present invention it will always be possible for a skilled artisan to arrive at the reference level meeting those requirements. In one embodiment the reference level is determined in reference samples from healthy individuals. The reference level in one embodiment has been predetermined in reference samples from the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in reference samples from a disease entity investigated. In particular, the reference levels are for CRP 1.51 mg/L, homocysteine 12.16 μmol/L, NT-proBNP 263 pg/ml.

In one embodiment the reference level is set to the median value as determined from the overall distribution of the values in a disease entity investigated. The reference level may vary depending on various physiological parameters such as age, gender or subpopulation, as well as on the means used for the determination of the biomarker Y referred to herein. In one embodiment, the reference sample is from essentially the same type of cells, tissue, organ or body fluid source as the sample from the individual or patient subjected to the method of the invention, e.g. if according to the invention blood is used as a sample to determine the level of biomarker Y in the individual, the reference level is also determined in blood or a part thereof.

In certain embodiments, the term “at the reference level” refers to a level of the biomarker in the sample from the individual or patient that is essentially identical to the reference level or to a level that differs from the reference level by up to 1%, up to 2%, up to 3%, up to 4%, up to 5%.

In certain embodiments, the term “greater than the reference level” refers to a level of the biomarker in the sample from the individual or patient above the reference level or to an overall increase of 5%, 10%, 20%, 25%, 30%, 40%, 50%, 60%, 70%, 80%, 85%, 90%, 95%, 100% or greater, determined by the methods described herein, as compared to the reference level. In certain embodiments, the term increase refers to the increase in biomarker level in the sample from the individual or patient wherein, the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100- fold higher as compared to the reference level, e.g. predetermined from a reference sample.

In certain embodiments, the term “decrease” or “below” herein refers to a level of the biomarker in the sample from the individual or patient below the reference level or to an overall reduction of 5%, 10%, 20%, 25%, 30%, 40%, 50%, 60%, 70%, 80%, 85%, 90%, 95%, 96%, 97%, 98%, 99% or greater, determined by the methods described herein, as compared to the reference level. In certain embodiments, the term decrease in biomarker level in the sample from the individual or patient wherein the decreased level is at most about 0.9-, 0.8-, 0.7-, 0.6-, 0.5-, 0.4-, 0.3-, 0.2-, 0.1-, 0.05-, or 0.01- fold of the reference level, e.g. predetermined from a reference sample, or lower.

In an other embodiment, the present invention may allow to reduce the sample size of secondary prevention trials as shown Table 3.

TABLE 3
reduction sample size of secondary prevention trials

| | | Absolute Sample |
|---|---|---|
| | | Size Reduction on dalO |
| | Relative Sample Size | (Sample size with enrichment/ |
| | Reduction Placebo | Sample size without enrichment) |
| | Estimation | Placebo Estimation |
| Minimum | 45.70% | 7254 (8618/15872) |
| Biomarker | | |
| (3 Marker) | | |
| Framingham | 27.90% | 4431 (11441/15872) |
| Reynolds | 24.40% | 3866 (12006/15872) |

The following example is intended merely to illustrate the practice of the present invention and is not provided by way of limitation.

### EXAMPLE 1

Dal-OUTCOMES trial (NC20971) was a double blind, randomized, placebo-controlled, parallel group, multi-center, phase III study to assess the safety and efficacy of the CETP inhibitor dalcetrapib in patients recently hospitalized for an Acute Coronary Syndrome (ACS). At time of the interim analysis the study included 15871randomized patients, distributed over two treatment arms: placebo (7933 patients) and dalcetrapib (600 mg daily; 7938 patients). The study has shown no evidence of reduction of the event rate in the primary efficacy endpoint in the dalcetrapib arm compared to the placebo arm. The dal-OUTCOMES study details can be found in G. Schwartz et al., N. Engl. J. Med.367; 22, 2012.

### Sample Collection

After overnight fast, blood was collected into SST tube, mixed by gentle inversion 5 times, followed by 30 min allowing sample to clot. Then centrifugation at 1500-2000 g (approx 4000-5000 rpm) for 15 minutes within 1 hour of collection. Serum was transferred into the plain cap transfer tube and immediately frozen.

Patient samples were assayed to evaluate the utility of several biomarkers to aid in assigning an increased likelihood of a subject of experiencing a subsequent cardiovascular event.

Samples obtained from each patient were analyzed by immunoassay to determine the level of each biomarker. Immunoassays were operated in a sandwich assay format or, for NT-proBNP, using the

Elecsys® proBNP platform (Elecsys® 20.10 immunoanalyzer).

The essays have been used for the respective biomarker are as follows:

NT-proBNP—Roche Immunoassay (Elecsys®)

High sensitive CRP—Roche Clinical Chemistry Assay; Cardiac C-Reactive Protein (Latex) High Sensitive on COBAS®

Homocysteine—Roche Clinical Chemistry Assay Roche Homocysteine enzymatic assay on COBAS ®.

Concentrations of homocysteine, “total homocysteine” was measured in serum samples using commercial assay kits according to the manufacturer's protocol.

Concentrations of CRP (High sensitive CRP) were measured in duplicate in serum samples using commercial enzyme-linked immunosorbent assay kits according to the manufacturer's protocol. Plasma samples were diluted X fold. The Lower Limit of Quantification was determined and set to X ng/ml.

NT-proBNP (cat. no. 04842464190, Roche Diagnostics, Mannheim, Germany) was measured serum using commercial CE certified test kits following the manufacturer's instructions.

The Following Characteristics were Evaluated:

Dynamic concentration range; Lower and upper limits of quantification; Matrix effects; Precision; Accuracy; Stability; Selectivity and specificity; Dilution parallelism; and Interfering agents.

### Sample Size

Biomarker measurements were performed on a selected subset of patients in order to enable a time and cost efficient. Given that only about 7% of the patients with available baseline serum samples experienced an event, it was possible to reduce the number of analyzed samples dramatically while retaining almost complete power by applying a Nested Case-Control (NCC) design. The NCC design included all patients with a primary composite endpoint (PCE) event or cardiovascular (CV) death and available serum samples. For each of the selected event patients matching control patients who were still at risk of experiencing an event at the respective event time are randomly selected. For the primary analysis population a 4:1 matching of controls to events was performed.

Sample size calculations were based on the log-rank test which is equivalent to the score test in a univariate proportional hazards regression with a dichotomic explanatory variable. As analyses did not include the whole ITT population but a subset selected by the chosen NCC design, power was reduced in comparison to the whole population. In the 4:1 matching case power was about 80% of the power on the complete ITT population. All reported power figures were corrected by the relative power reduction due to the NCC design.

In the placebo group, there were 476 observed PCE events. With the chosen 4:1 matching this gave a 70% power to detect a risk reduction of 25% and with 50% power for detecting a risk reduction of 19%. Numbers refer to a two-sided Type I error of 5%.

### Statistical Analysis

### Analysis Populations

Results were based on the second interim analysis of the phase 3 clinical trial NC20971. At time of the interim analysis the study included 15872 randomized patients, distributed over two treatment arms: placebo (7934 patients) and dalcetrapib (600 mg daily; 7938 patients). The study did not show any evidence of reduction of the event rate in the primary efficacy endpoint in the dalcetrapib arm compared to the placebo arm.

### Intent-to-Treat Population (ITT)

All patients randomized, excluding those identified as having fraudulent data impacting the interpretability of the study or erroneously randomized by the investigator without any intention to treat them, were included in the intent to treat population.

The ITT population included 15871 patients, 1135 experienced a PCE event and 1101 experienced a DMS event (a CHD death, non-fatal MI or stroke event). All further selected analysis populations were based on the ITT population.

### Automated Assay Populations

All patients from the ITT population with a PCE event or a CV death and available baseline serum samples and corresponding controls were selected by the 4:1 nested case-control matching. Because case-control matching was performed by random sampling with replacement it is likely that samples were selected more than one time for different risk sets. Thus some patient samples are listed several times in the analysis data set but only measured once.

Corresponding to the two analysis endpoints we defined two different populations:
Automated assay PCE population—contains all measured PCE events and matching controls
Automated assay DMS population—contains all measured DMS events and matching controls.

Baseline serum samples were available from 961 PCE and 851 DMS events. With the 4:1 matching used for automated assays this led to a total number of 4805 patients (4112 without duplicates) in the automated assay PCE population and 4255 patients (3712 without duplicates) in the automated assay DMS population. The automated assay PCE population was the primary analysis population.

### Pre-Processing of Biomarker Data

### Handling of Truncated Values

Biomarker measurements were provided in the format specified in the File Format Specification (FFS) document. Data files contained the actual sample measurements and information on the measurement unit (e.g. ng/ml). Values below the measurement range were reported as “<Y”, where Y is the lower limit. Values above the measurement range were reported as “>X”, where X is the upper limit.

The measurement data were pre-processed as follows:
1. “<Y” entries was replaced with Y/2
2. “>X” entries was replaced with X

Biomarkers with more than 70% truncated values were excluded from the analysis.

### Handling of Missing Values

Biomarkers with more than 20% missing values were excluded from the analysis. For regression analysis remaining missing values were replaced with the median of the respective biomarker in the data matrix.

### Transformation and Change Calculation

All biomarker measurements were log2 transformed. For the analysis of concentration changes the relative log2 change between baseline and month 3 visit were calculated as log2(V3/BL).

### Feature Reduction

The set of biomarkers assessed in this study contained several assays that assessed the same analyte or assess sub-components of other analytes (e.g. LDL-c and sdLDL). It was expected that many of these biomarker pairs show very high correlations, which reduce statistical power and might cause problems in estimation of prediction models and feature selection.

It was identified highly correlated groups of biomarker candidates by hierarchical clustering using the Spearman correlation coefficient as similarity measure. If clusters of highly correlated proteins were identified these clusters were reduced to one single variable for primary analysis. Reduction was achieved by selecting one representative biomarker for the cluster—either based on preference (e.g. analytical assay performance or biological reasoning) or based on the smallest distance to all other cluster members.

### Analysis of Biomarker and Treatment Group Comparability

### Demographics

Summary statistics are presented for both treatment groups within the complete study ITT, the automated assay PCE, and the manual assay PCE population for the following demographics and baseline characteristics: sex, age, race, ethnicity, geographic region, weight, height, and body mass index (BMI).

### Baseline Disease Characteristics

Summary statistics are presented for both treatment groups within the complete study ITT, the automated assay PCE, and the manual assay PCE population for the following baseline disease characteristics: hypertension, diabetics, metabolic syndrome, previous MI/UA and geographic origin.

### Missing Data

For regression analysis missing values of continuous baseline characteristics and biomarker values were replaced with the median of the respective variable in the data matrix. For categorical baseline variables the most frequent category were imputed.

Endpoint imputation was not necessary since all used statistical methods are able to handle censored data.

### Measures of Prediction Accuracy

### Absolute Measures

We used the following measures of model fit in order to assess the accuracy of a model for predicting the risk of a future cardiovascular event:

### Concordance Index

The concordance index C was defined as:

C═Pr[Z₁≅Zⱼ|Dᵢ=1,tᵢ<tⱼ],

where Z₁ is the prediction model risk score, t₁ the survival time, and Dᵢ an indicator variable for the observation of an event for the i^{th} patient (Pencina, M. J. (2008)., Statistics in Medicine, 27(2), pp. 157-172.)(Chambless, L. E. et al. (2011). Statistics in Medicine, 30(1), pp. 22-3 8)

The C index is a non-parametric estimator of the proportion of all patient pairs for which model prediction and observed outcome are concordant.

### Time Dependent AUC

In the analysis of time to event data the classic diagnostic measures of sensitivity, specificity and area under the ROC curve (AUC) become functions of time. For a given set of patients that is at risk of experiencing an event at a given point in time these measures quantify the discrimination between imminent cases and near term controls based on a given risk score or biomarker and a cutoff value.

The time dependent AUC was defined as:

AUC(t)═Pr[Zᵢ>Zⱼ|Dᵢ=1,tᵢ=t,tᵢ<tⱼ],

where Zᵢ is the prediction model risk score, t is the point in time of interest, tᵢ the survival time, and Dᵢ an indicator variable for the observation of an event for the i^{th} patient (Saha-Chaudhuri, P. et al. (2012). Non-parametric estimation of a time-dependent predictive accuracy curve. Biostatistics. AUC(t) is a non-parametric estimator of the proportion patient pairs for which model prediction and observed outcome are concordant within a defined time frame of interest.

### Relative Measures for Model Comparison

We used the following measures for comparison of two models with regard to prediction accuracy:

### Difference of Concordance Indices

The difference between the C indices of two models is a non-parametric measure for improvement of model prediction accuracy.

### Difference of Time Dependent AUC Values

The difference between the AUC(t) of two models is a non-parametric measure for improvement of model prediction accuracy at a given point in time.

### Deviance

The deviance compares the fit of two nested parametric models based on the likelihood ratio. The deviance was defined as:

D(y)=−2(log(p(y|\hat{θ}₀))−log(p(y|\hat{θ}₁))),

where y is the observed data, and \hat{θ}₀ and \hat{θ}₁ are the estimated parameters of the baseline and the full model respectively. Since the deviance is directly based on the model likelihoods it is directly linked to the optimization criterion used for model fitting. It has the disadvantages of depending on model calibration and not having any clinical interpretation.

### Prognostics Biomarker Discovery

Objective of the prognostic biomarker discovery was the identification of a set of biomarkers which improve the risk prediction for a patient significantly. Identified biomarkers should add additional information to well established risk markers (e.g. HDL, LDL) and other prognostic factors (e.g. diabetes or smoking).

### Primary Analysis

The primary analysis was performed on the primary composite endpoint. The analysis data set contained all patients selected for PCE risk sets. The primary analysis was not stratified by risk sets; each selected patient entered the analysis only once. A stratified analysis was performed for the final selected model as a sensitivity analysis to check for a potential bias of the primary analysis as described herein.

Potential explanatory variables for this analysis were the baseline concentrations of laboratory and automated assay biomarkers listed in tables 8 and 9 and demographic variables as well as clinical variables associated with health status and clinical records at baseline.

The primary analysis was performed on the placebo group of the automated assay PCE population as defined herein. The analysis population was limited to the placebo group in order to represent a standard of care treatment scenario.

The primary analysis encompassed the selection of an optimal prognostic model and the determination of the risk prediction performance of the selected model. This was performed by a nested cross-validation analysis on the placebo group (inner cross-validation for determination of optimal model complexity, outer cross-validation for determination of risk prediction performance). In addition we determined the risk prediction performance of the model on the treatment group. Assuming complete inefficacy of dalcetrapib, this would provide risk prediction performance estimates on an independent patient cohort.

### Model Selection

Time to event was modeled with an unstratified Cox proportional hazard model. Variable selection was performed by the LASSO method (Tibshirani, R. et al. (1996). J. Royal. Statist. Soc B., 1, pp. 267-288), using the pathwise cyclical coordinate descent method for Cox regression (Simon, N. et al. (2011). Journal of Statistical Software, 39(5), pp. 1-13).

In order to assess the value of additional information delivered by the new biomarkers it was necessary to select two models. The first model was selected from all non-biomarker variables and well established CV risk biomarkers. The second model was based on the first model but extended with the novel biomarker candidates. The first model was called “reference model” and the second model “biomarker model”. The reference model was created first and based on the reference model the biomarker model was built. The biomarker model included all variables selected by the reference model.

X₁, . . . , Xₙ represent all non-biomarker and established CV risk biomarker variables and Z₁, . . . , Zₘ all novel biomarker variables. A represents the index set of size k of all non-biomarker and established CV risk biomarker variables selected by LASSO. Thus A was defined as
A={i |Xᵢ is selected for prognostic model},
thus X_{A1}, . . . , X_{Ak} were all selected non-biomarker and established CV risk biomarker variables. Then the reference model is:

Y=X_{A1}+ . . . +X_{Ak}.

Based on the reference model the LASSO procedure could then in a second step select from the novel biomarker candidates Z₁, . . . , Zₘ further variables for inclusion in the model. B represents the index set of size p of all novel biomarker candidate variables selected by LASSO in addition to the reference model:

B={i|Zᵢ is a prognostic biomarker included in the prognostic model}.
Then the sum of X_{A1}, . . . , X_{Ak} and Z_{B1}, . . . , Z_{Bp} constitute the prognostic biomarker model:

Y=X_{A1}+ . . . +X_{Ak}+Z_{B1}+ . . . +Z_{Bp}.

In the LASSO procedure the complexity (number of selected features) of the model is regulated by the penalization parameter lambda. Large lambdas (high penalization) lead to few included variables and small lambdas (low penalization) to more included variables. For the selection of the optimal lambda a 10-fold cross validation was used. This means the data set was split into 10 (equal sized) parts: 9 were used for training, the 10th was used for testing. Then the training/testing set assignment was permutated, so that each of the 10 parts belongs 9 times to the training set and one time to the test set. On each test set outcome was predicted for each lambda and compared with the actual outcome. Model prediction quality was assessed for each lambda by c-index and stored. The described k-fold cross-validation procedure was repeated 5 times on randomly ordered sample sets. For each lambda prediction quality was summarized by the median of the cross-validation results (10 * 5=50 c-indexes on the test set). The lambda with the maximal corresponding median was selected and called λₒₚₜ. Because this “minimal loss” λₒₚₜ often leads to complex models that show no significant or clinically meaningful improvement over less complex models, an additional correction step was included. We defined a significant improvement in c-index as one standard deviation of the observed cross-validation c-indices at λₒₚₜ. We defined a clinically meaningful improvement in c-index as a delta of 0.0025 (corresponding to 0.25% of patient pairs showing an increased concordance). This number was derived from published c-indices of established risk factors like HDL (ΔC=0.013-0.023) or smoking (ΔC=0.006-0.024) (Chambless, L. E. et al. (2011). Statistics in Medicine, 30(1), pp. 22-38).

The selected lambda was:

λₛₑₗ=max(λ| median(c-index [λ])>=median(c-index [λₒₚₜ])−max(sd(c-index[λₒₚₜ]), 0.0025))

This means we took the largest lambda (smallest model) where the performance (median of c-index) was greater or equal as the performance of the λₒₚₜ model minus its standard deviation. If the standard deviation was below 0.0025 then 0.0025 was subtracted. The standard deviation sd(c-index[λₒₚₜ]) was estimated from the cross-validation runs.

The final model was selected using LASSO with lambda λₛₑₗ. The regression coefficients of the final model were estimated by unpenalized Cox regression.

First the reference model was built according to the described LASSO procedure. Then the biomarker model was built using the same LASSO procedure to add additional biomarkers. The only difference in the second LASSO phase is that all coefficients of the variables selected in the reference model are not penalized and therefore are always included in the model disregarding the lambda.

The two models were then be compared in their ability to predict the patient response.

### Generalization Performance

Because model selection was performed on the same data set as evaluation of the model fit a comparison of two models with regard to model fit would prefer the more complex model. Hence an outer cross validation step was necessary.

This means before the data gets into the “internal” cross-validation step described above, data was split in an outer test and training set. The training set included 80% of the cases. The cases included in the training set were selected randomly (Monte Carlo cross-validation). The test set included all not-selected cases. Then the approach described in “model selection” was applied on the outer training set. As a result we received two models, the reference model and the biomarker model. Based on these two models prediction over the response variable was done on the test set and the c-index calculated for each model. Difference in prediction quality between the reference model and the biomarker model were assessed by the absolute difference in c-index

c-index_{Diff}=c-index_{Biom}−c-index_{Ref}. The outer cross-validation step was repeated at least 100 times.

The median of c-index_{Diff} from all cross-validation runs is an unbiased measure for the improved model performance by inclusion of the new biomarkers. Confidence intervals for average model fit improvement are estimated from the 90% sample quantiles of the cross-validation results. The use of the identified biomarker panel results in a significant improvement of risk estimation if the lower bound of the calculated cross-validation confidence interval is above zero.

### Model for Calculation of Absolute Risk:

The general form of the Cox model is: h(t)=h0(t)*exp(b1*X1+b2*X2+. . . ), where b equals the natural logarithms of the HR values. In our model X1 is equal to age, X2 is equal to pulse rate, X3 is equal to LDL, . . .

The HR of age is 1.004, the natural logarithm is 0.004, which can be found in Table 4, row 1, column coef. The HR of pulse rate is 1.016, the natural logarithm is 0.016 (see Table 4, row 2, column coef).

h0(t), the baseline hazard ration (baseline HR), is a time-dependent function that does not contain information from the variables that are included in the risk model.

TABLE 4
Summary statistics of multivariate prognostic model (Mininun Biomarker
Model). Analysis population is the automoated analysis population.
Number of observations: 2080\hat{ }2 (including 489 events) C index: 0.7081.

| | coef | exp(coef) | se(coed) | z | Pr(> \|z\|) |
|---|---|---|---|---|---|
| Age | 0.004 | 1.004 | 0.005 | 0.689 | 0.491 |
| Pulse rate | 0.016 | 1.016 | 0.004 | 3.581 | 0.000 |
| LDL cholesterol | 0.285 | 1.330 | 0.099 | 2.886 | 0.004 |
| Diabetes = “YES” | 0.102 | 1.107 | 0.101 | 1.000 | 0.317 |
| Peripheral vascular | 0.361 | 1.435 | 0.126 | 2.865 | 0.004 |
| disease = YES | | | | | |
| Congestive heart | 0.028 | 1.028 | 0.113 | 0.244 | 0.807 |
| failzure = YES | | | | | |
| Previous | 0.328 | 1.388 | 0.136 | 2.419 | 0.016 |
| revascularization = YES | | | | | |
| Coronary heart disease | 0.317 | 1.374 | 0.187 | 1.699 | 0.089 |
| before index event = YES | | | | | |
| Previous ACS = YES | 0.282 | 1.326 | 0.172 | 1.642 | 0.101 |
| Diuretic therapy = YES | 0.284 | 1.329 | 0.106 | 2.669 | 0.008 |
| High sensitive CRP | 0.055 | 1.056 | 0.029 | 1.858 | 0.063 |
| Homocysteine | 0.230 | 1.259 | 0.096 | 2.389 | 0.017 |
| NT-proBNP | 0.168 | 1.183 | 0.030 | 5.681 | 0.000 |

The prognostic model is illustrated in Table 5.

Biomarkers are entered as continuous variables into the model. HRs are given per log step—in this case to the base 2. In case of homocysteine (HR 1.259) this means that risk is increased by 25.9% per log step respectively per doubling of homocysteine levels. The respective numbers for NT-proBNB are 18.3% per log step (doubling) and for hsCRP 5.6% per log step (doubling).

The same applies to age, pulse rate, and LDL, while the presence of Diabetes (HR 1.107), peripheral vascular disease (HR 1.435), congestive heart failure (HR 1.028), previous ACS (HR 1.326), previous revascularization (HR 1.388), coronary artery disease before index event (HR 1.374), and treatment with diuretics (HR 1.329) increase risk by 10.7%, 43.5%, 2.8%, 32.6%, 38.8%, 37.4%, and 32.9%.

TABLE 5
Prognostic model

| | Minimum Biomarker Model |
|---|---|
| Risk Factor | HR (95% CI) |
| Age - years | 1.004 (0.993-1.014) |
| Pulse rate - BPM | 1.016 (1.007-1.025) |
| LDL - log₂ ng/ml | 1.330 (1.096-1.615) |
| Diabetes | 1.107 (0.907-1.350) |
| Peripheral vascular disease | 1.435 (1.121-1.837) |
| Congestive Heart Failure | 1.028 (0.823-1.284) |
| (CHF Class I or Class II) | |
| Previous acute coronary syndrome | 1.326 (0.947-1.856) |
| (MI or unstable angina) | |
| Previous revascularization (PCI, CABG), | 1.388 (1.064-1.811) |
| previous stroke or TIA | |
| Coronary Heart Disease before IE | 1.374 (0.952 1.981) |
| Treatment with diuretics | 1.329 (1.078-1.637) |
| CRP | 1.056 (0.997-1.119) |
| Homocysteine | 1.259 (1.042-1.521) |
| ProBNPII | 1.183 (1.116-1.254) |

TABLE 6
Prognostic Model Performance

| | Placebo | | Placebo | |
|---|---|---|---|---|
| | (Resubstitution) | | (Cross Validation) | |
| Prognostic Model | C-index | Royston D | C-index | Royston D |
| Framingham | 0.616 | 1.91 | — | — |
| | (0.593-0.638) | (1.69-2.16) | | |
| Reynolds | 0.609 | 1.88 | — | — |
| | (0.587-0.631) | (1.66-2.13) | | |
| Minimum | 0.708 | 3.53 | 0.697 | 3.26 |
| Biomarker | (0.682-0.734) | (3.03-4.10) | | |
| (3 Markers: | | | | |
| Homocysteine, | | | | |
| CRP, NT-proBNP) | | | | |

Results of cross-validation of the described model according to the method described above

TABLE 7
Performance of established vs. novel models

| | | ΔC-Index vs. | | NRI vs. |
|---|---|---|---|---|
| | C-Index | Framingham | Royston D | Framingham |
| Framingham | 0.616 | — | 1.91 | — |
| Reynolds | 0.609 | −0.007 | 1.88 | +0.011 |
| Minimum | 0.708 | +0.092 | 3.53 | +0.337 |
| Biomarker | | | | |

Royston D = Approximation of hazard ratios for multivariate score median cutoff
NRI = Net Reclassification Improvement

TABLE 8
Prognostic Model Comparison,

| | | | | | | NRI vs. |
|---|---|---|---|---|---|---|
| | | | | | | Reference |
| Prognostic | | Event | Risk Category (at 2 years) | | | Model |
| Model | Arm | Category | ≦3.6% | 3.6-7.7% | >7.7% | (at 2 years) |
| Framingham | Placebo | Controls | 20% | 43% | 37% | −0.260 |
| | | | (166/824) | (355/824) | (303/824) | |
| | | Cases | 13% | 34% | 53% | |
| | | | (59/455) | (153/455) | (243/455) | |
| Reynolds | Placebo | Controls | 16% | 33% | 43% | −0.249 |
| | | | (135/824) | (275/824) | (353/824) | |
| | | Cases | 14% | 28% | 57% | |
| | | | (65/455) | (129/455) | (261/455) | |
| Minimum | oPlacebo | Controls | 46% | 32% | 21% | 0.077 |
| Biomarker | | | (380/824) | (267/824) | (177/824) | |
| (3 Markers: | | Cases | 16% | 30% | 54% | |
| homcysteine, | | | (73/455) | (135/455) | (247/455) | |
| proBNP, CRP) | | | | | | |

TABLE 9
Biomarkers measured on automated assays. Established CV risk markers.

| No. | Marker | Supplier |
|---|---|---|
| 01 | HDL-C | Roche |
| 02 | LDL-C | Roche |
| 03 | Triglycerides | Roche |

TABLE 10
Biomarker measured on automated assays. Potential new CV risk markers.

| No. | Marker | Supplier |
|---|---|---|
| 01 | Cystatin C | Roche |
| 02 | Uric acid | Roche |
| 03 | Homocysteine | Diazyme |
| 04 | hsCRP | Roche |
| 05 | Total Cholesterol | Roche |
| 06 | Apo-A1 | Roche |
| 07 | ApoB | Roche |
| 08 | Lp(a) | Denka |
| 09 | PON-1 activity | Roche |
| | | (R&D) |
| 10 | Remnant particles | Kyowa |
| 11 | HDL-C3 | Kyowa |
| 12 | sdLDL | Denka |
| 13 | HDL-C3 | Denka |
| 14 | Adiponectin | Denka |
| 15 | ApoA-II | Kamiya |
| 16 | ApoC-III | Kamiya |
| 17 | GDF-15 | Roche |
| | | (R&D) |
| 18 | hsTnT | Roche |
| 19 | NTproBNP | Roche |
| 20 | IL-6 | Roche |
| 21 | C-Peptide | Roche |
| 22 | Insulin | Roche |

1. A method for identifying a subject suffering from stable CHD, particularly with a documented recent Acute Coronary Syndrome (ACS), more particularly a post Acute Coronary Syndrome (ACS), most particularly a recent ACS as having an increased risk of a cardiovascular event, particularly an other cardiovascular event, more particularly a secondary cardiovascular event, the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

2. The method according to claim 1, wherein the cardiovascular event is selected from cardiovascular death, non-fatal myocardial infarction (MI), non-fatal stroke of ischemic origin, hospitalization for unstable angina and coronary revascularization and cardiac arrest.

3. The method according to claim 1, wherein the detecting comprises contacting, in vitro, the sample with a combination of detection agents, each agent having specific binding affinity for one of the biomarkers.

4. The method according to claim 3, wherein the agent is an antibody or fragment thereof.

5. The method according to claim 1, wherein the sample is a serum or a blood sample.

6. The method according to claim 1, wherein the subject is identified as having an high risk of a cardiovascular event, particularly an other cardiovascular event, more particularly a secondary cardiovascular event, when the amounts of the NT-proBNP, homocysteine and CRP in the sample are greater than the median of their respective reference amount.

7. The method according to claim 1, wherein the subject is identified as having an low risk of a subsequent cardiovascular event when the amounts of the NT-proBNP, homocysteine and CRP in the sample is lower than the median of their respective reference amount.

8. The method according to claim 1, wherein the subject is identified as having an increased risk of a cardiovascular event, particularly an other cardiovascular event, more particularly a secondary cardiovascular event, when the amount of the NT-proBNP, homocysteine and CRP in the sample is in the fourth quartile range of their respective reference amount.

9. The method according to claim 1, further comprising the step of recommending a therapy to treat cardiovascular disease, if the subject is identified as having an increased risk of a cardiovascular event, particularly an other cardiovascular event, more particularly a secondary cardiovascular event.

10. The method according to claim 1, further comprising the step of administering to the subject a pharmaceutical agent to treat cardiovascular disease, if the subject is identified as having an increased risk of cardiovascular event, particularly an other cardiovascular event, more particularly a secondary cardiovascular event.

11. The method according to claim 9, wherein the therapy comprises an investigational new drug therapy.

12. A device adapted for carrying out the method according to claim 1 comprising:
a) an analysing unit comprising a combination of detection agents which specifically bind to NT-proBNP, homocysteine and CRP, the analysing unit adapted for contacting, in vitro, the sample from the subject with the detection agent;
b) an evaluation unit including a computing device having a database and a computer-implemented algorithm on the database, the computer-implemented algorithm when executed by the computing device determines an amount of the biomarker in the sample from the subject and compares the determined amount of NT-proBNP, homocysteine and CRP with the corresponding NT-proBNP, homocysteine and CRP reference amount and provides a diagnosis of at increased risk of a cardiovascular event, particularly an other cardiovascular event, more particularly a secondary cardiovascular event, if the amount of the NT-proBNP, homocysteine and CRP determined in the step of determining is greater than the corresponding NT-proBNP, homocysteine and CRP references amount.

13. The device of claim 12, wherein the database further includes the NT-proBNP, homocysteine and CRP references amount.

14. A kit adapted for carrying out the method according to claim 1, comprising detection agents for NT-proBNP, homocysteine and CRP and instructions for carrying out the method.

15. The kit of claim 14 further comprising a combination of detection agents for NT-proBNP, homocysteine and CRP.

16. A method for identifying a subject as having an increased risk of experiencing a cardiovascular event, particularly an other cardiovascular event, in particular a secondary cardiovascular event, following an acute coronary syndrome, more particularly following a recent acute coronary syndrome, the method comprising:
a) detecting the amount of N-terminal of the pro-hormone brain natriuretic peptide (NT-proBNP), homocysteine and C-reactive protein (CRP) in a sample of a subject;
b) comparing the amount of NT-proBNP, homocysteine and CRP to a reference amount of NT-proBNP, homocysteine and CRP; and
c) identifying the subject as having an increased risk of a cardiovascular event, particularly an other cardiovascular event, more particularly a secondary cardiovascular event, if the amount of NT-proBNP, homocysteine and CRP in the sample is greater than the reference amount of NT-proBNP, homocysteine and CRP.

17.-32. (canceled)

33. A method according to claim 1, wherein the reference amounts are for CRP 1.51 mg/L, homocysteine 12.16 μmol/L and NT-proBNP 263 pg/ml.

34. (canceled)


SEQUENCE LISTING (2 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP